(19) United States
(12) Patent Application Publication
(10) Pub. No.: US 2007/0128668 A1
(43) Pub. Date: Jun. 7, 2007

(54) Quantitative use of tracer cells

(76) Inventor: Larry Burg, Framingham, MA (US)

Correspondence Address: Theodore Allen, Esq.;Cytyc Corporation, 250 Campus Drive, Marlborough, MA, 01752, US

(21) Appl. No.: 11/291,723

(22) Filed: Dec. 1, 2005

(51) Int. Cl. G01N 33/567

(52) U.S. Cl. 435/7.2

(57) ABSTRACT

A method is provided for the quantitative assessment of cell deposition onto a planar surface. In particular, the present invention provides a method of measure the quantitative transfer of labeled tracer cells to a glass slide to evaluate the effectiveness of and uniformity of cell transfer to the surface of the slide.

### FIELD OF THE INVENTION

A method is provided for the quantitative assessment of cell deposition onto a planar surface. In particular, the present invention provides a method of measure of the quantitative transfer of labeled tracer cells to a glass slide to evaluate the effectiveness of and uniformity of cell transfer to the surface of the slide.

### BACKGROUND OF THE INVENTION

The method of the present invention generally relates to the field of transferring cells to a planar surface. In particular, the invention provides a method for collecting cells from a biological sample, adding labeled tracer cells, transferring the cells onto a glass slide, and determining the effectiveness and uniformity of the cell transfer. The invention is useful in cytology, which is a medical and laboratory science that makes diagnoses based on visual assessment of cellular characteristics. A typical cytological technique is a “pap smear” test, which is used, in one instance, to detect abnormal cells in a woman's cervix before they develop into cancer cells. The invention can also be used in applications where cells are transferred from a liquid suspension onto a planar surface (e.g., a glass slide) such as in-situ hybridization and immunocytochemistry. Cytology is less invasive to a patient than traditional surgical pathological procedures, e.g. biopsy. All that is required for cytology is that a sample of cells be obtained from the patient, which can typically be done by scraping or swabbing an area, as in the case of cervical samples, by washing a body site as in bronchial or breast ductal lavage, by procuring fluids from body sites such as the chest cavity, bladder, or spinal canal, or by fine needle aspiration. Typically, cells obtained by one of these methods are placed into a solution to preserve cellular morphology and other characteristics, the cells are collected from the solution, transferred onto a glass slide and stained for microscopic viewing. This processing of the cells typically requires that the cells be separated from one another, i.e. dispersed, so that individual cells can be transferred to the glass slide for visual examination. Diagnostic accuracy by cytological evaluation depends on having a sufficient number of cells for inspection and displaying those cells in a manner that facilitates inspection of cellular features (i.e. a thin layer of cells distributed uniformly on the surface). The phrase “inspection” is used herein in a broad context and includes human visual examination as well as instrument image assessment. It also includes inspection with various types of illumination and signal detection techniques. This invention provides a tool that generates objective quantitative information on both of these essential requirements; cell numbers and their distribution on a planar surface.

The transfer of cells to a glass slide was historically performed by simply streaking the specimen collection device onto the slides or centrifuging cells onto the slides. Such techniques have several shortcomings. These shortcomings include inter-sample contamination and poor reliability and repeatability which may lead to diagnostic inaccuracies and imprecision. In particular, these methods do not control cell numbers or distribution so that the resulting slides may contain too many or too few cells and the cells may be layered upon each other and thereby obscure inspection. These shortcomings are increasingly significant with the increasing use of cytologic diagnosis. More recently, liquid-based cytology methods have been developed that largely overcome these shortcomings. In liquid-based methods, cells are first placed in a solution that preserves cellular structure and then dispersed to generate a homogeneous cell mixture. In one technique, a controlled number of cells are collected by aspiration onto a filter membrane, the number of cells reproducibly obtained by continuously measuring the flow rate through the filter. The distribution of cells is also controlled, as the filter is designed with a uniform distribution of pores across its surface, which allows uniform fluid flow, and thus cell collection, across the entire surface. The cells are then transferred to the glass slide by pressing the filter to the glass surface. Although generally reliable, this process may be disrupted by biological components present in excess in certain clinical specimens, such as blood or mucus. Thus, there exists a need for a quantitative assessment of the effectiveness of the transfer of cells to a slide and for determining both the number of cells and their distribution on the slide.

Methods for labeling homogeneous cell preparations and using them as controls to monitor cell-based tests have been frequently described. For example, cells have been radioactively labeled and injected into animals to study their fate using imaging technology. Cells have been labeled to study flow characteristics in restricted channels, such as the study of blood flow and thrombosis. Fluorescently labeled tracer cells have been used to study plankton growth and grazing rates using flow cytometry. Similarly, flow cytometry in conjunction with fluorescence in-situ hybridization (FISH) has been used with internal control cells to measure telomere lengths. There are a number of commercially available fluorescent probes and microspheres that may be used as tracers of cell morphology and fluid flow (e.g. as reference standards for flow cytometry) (Molecular Probes, Inc., Eugene, Oreg.). However, the use of pre-labeled tracer cells to quantify the deposition of cells onto planar surfaces and/or determine the uniformity of cell distribution has not been previously described.

An ideal tool for cellular applications would be comprised of tracers that are of biological origin (cells) and thus closely mimic other cells (as opposed to the use of some artificial tracer such as a micro-bead) and would be quantitative (thus subjective to statistical analyses), objective, robust, accurate and reproducible. Furthermore, the ideal tracers would use a labeling methodology that does not alter surface properties that could interfere with deposition onto surface (as opposed to decorating cell surface with antibodies for example) and would permit measurement of total cell deposition and uniformity.

Thus, the present invention describes a quantitative assessment of cell deposition onto a planar surface or any other surface (e.g. well of a microtiter plate). The present invention also describes a method capable of measuring the quantitative transfer of labeled tracer cells to a glass slide to evaluate the effectiveness of and uniformity of cell transfer to the surface of the slide.

Other objects of the invention will in part be obvious and will in part appear hereinafter.

### BRIEF DESCRIPTION OF THE FIGURES

FIG. 1. is an illustration of the reproducibility of the transfer of tracer cells to a plurality of slides made in succession.

### DEFINITIONS

As used herein, the following terms have the meanings ascribed to them unless specified otherwise.

The term “labeling” refers to the process of labeling a cell or cells with dyes, (e.g., chromogenic or fluorescent dyes), stains, reagents (e.g. chemiluminescent or immunological reagents), or other detectable moieties (e.g. radioactive isotopes, nucleic acid probes, binding partners, micro-particles), such that attachment onto the cell surface or incorporation into a cell of such reagents, stains, dyes, or other labels allows for the monitoring of the presence or absence of such labeled cell(s) in a sample of unlabeled cells. The term “labeling” also encompasses the labeling of a cell or population of cells with a multitude of different types of labels, for example a nuclear stain plus a biotin-labeled or fluorescence-labeled antibody to a specific cell surface receptor. It is also anticipated that different labels may be applied to different cells of populations of cells within a single sample.

The term “tracer cell” refers to a labeled cell or a group of labeled cells used to indirectly monitor the behavior of cell(s) of interest without affecting the behavior of the cells of interest. Tracer cells may be derived from any homogeneous population of cells, whether purified directly from a biological source (tissue, blood, etc.) or from cells cultured in the laboratory (either primary or immortal cell lines; bacterial cultures). Depending on the application, tracer cells may be derived from any eukaryotic or prokaryotic biological source, including human, mammalian, non-mammalian, plant, bacterial, fungal, etc.

The term “planar surface” refers to a surface that is typically flat and two-dimensional such as the glass surface of a microscope slide or the plastic surface on the bottom of a well of a flat-bottom microtiter plate. In addition, the term refers to other non-flat surfaces to which cells may be deposited and subsequently detected, such as the surfaces of a conical tube or the well of a rounded-bottom microtiter plate. Such surfaces may either be native or modified with chemical or non-chemical treatments to impart specific surface characteristics (e.g. modification of surface charge on glass by treatment with poly-lysine or other adducts; etching of plastic to alter the surface texture; etc.).

The term “sample cells” refers to any cells derived from tissues from a living or dead biological source, including, for example, human peripheral blood or bone marrow, plasma, serum, cervical swab samples, biopsy tissue including lymph nodes, respiratory tissue or exudates, gastrointestinal tissue, urine, feces, semen or other body fluids, tissues or materials. In one embodiment, the sample cells are derived from a cervical scraping collected for liquid-based cytology for cervical cancer screening.

### SUMMARY OF THE INVENTION

The present invention generally relates to a method for the quantitative assessment of cell deposition onto a planar surface. In particular, the present invention provides a method of measure of the quantitative transfer of labeled tracer cells to a glass slide to evaluate the effectiveness of and uniformity of cell transfer to the surface of the slide. The method provides a means to quantify these parameters and provides a basis of standardization for comparing results between experiments or samples. In one embodiment of the present invention, a method for determining the deposition of cells onto a planar surface is presented, such method comprising labeling tracer cells, adding the labeled cells to a collection of non-labeled cells or sample cells that are in liquid suspension, dispersing the sample cells onto a planar surface and determining the effectiveness of transfer of the sample cells by determining the number of labeled cells transferred to the planar surface.

In another embodiment of the present invention, a method for determining the effectiveness and uniformity of cells deposited onto a microscope slide is presented, such method comprising labeling tracer cells, adding the labeled cells to a collection of non-labeled cells or sample cells that are in liquid suspension, dispersing the sample cells onto to a microscope slide, and determining the effectiveness of transfer of the sample cells by determining the number of labeled cells transferred to the microscope slide.

In yet another embodiment of the present invention, a method for determining the focal plane of a planar surface is presented, such method comprising labeling tracer cells, adding the labeled cells to a collection of non-labeled cells or sample cells that are in liquid suspension, dispersing the sample cells onto to a planar surface, and determining the focal plane of the planar surface by determining the location of the labeled cells on the planar surface.

In still another embodiment of the present invention, a method for determining the functional properties of a surface is presented, such method comprising the labeling of a plurality of types of tracer cells distinguishable one from the other (e.g. by differential labeling methods or physical feature such as size), adding the labeled cells to a collection of non-labeled cells or sample cells that are in liquid suspension, dispersing the sample cells onto to a planar surface, and determining which types of tracer cells are present on the surface.

### DETAILED DESCRIPTION OF THE INVENTION

The method of the present invention describes measuring the quantity and distribution of cells applied to planar surfaces, primarily glass microscope slides. One principle use of cells adhered to glass is in the field of cytology, and more precisely, liquid-based cytology. However, other slide-based diagnostic formats make use of cells applied onto microscope slides, including in-situ hybridization, immunocytochemistry and immunohistochemistry of thin-sections of tissue or cells imbedded in paraffin. These latter methods may employ a variety of detection techniques, such as direct labeling or the use of enzyme/substrate reactions and with chromogenic, fluorescent, radioactive or other labels.

Within the field of liquid-based cytology, it is important to apply an adequate number of cells in a uniform thin layer onto glass microscope slides (either coated or non-coated) to facilitate the morphological evaluation of those slides by trained cytotechnologists or cytopathologists. The ThinPrep® Pap Test (Cytyc Corporation, Marlborough Mass.) is a semi-automated system that produces a thin cell layer on slides from cervical specimens in order to screen for cervical cancer or morphological pre-cancerous cellular changes indicative of a high risk of progression to cervical cancer. Tracer cells can be used to quantitatively measure the effectiveness of transferring cells from the cervical specimens (collected in a cell preservative solution) to the microscope slide by the ThinPrep® system and to measure the uniformity of cell distribution on the glass surface. Within this context, one embodiment of the present invention is to use tracer cells derived from patients with cervical cancer. Such cells are readily available from the American Type Tissue Collection (ATCC) as immortal cultured cell lines and include CaSki, SiHa, SW756, C-4I, and other cell lines. These cells are ideal for use as tracer cells because they can be grown and purified in large numbers in the laboratory, are relatively uniform in size/morphology, are easily quantified and are expected to mimic abnormal cervical cells present in a clinical specimen.

Tracer cells may be derived from any homogeneous population of cells, whether purified directly from a biological source (tissue, blood, etc.) or from cells cultured in the laboratory (either primary or immortal cell lines). Depending on the application, tracer cells may be derived from any eukaryotic or prokaryotic biological source, including human, mammalian, non-mammalian, plant, bacterial, fungal, etc. The tracer cells may be labeled before use with those methods known to one skilled in the art (e.g., cytological stains, radioactive labeling, antigen-specific immunostaining, nucleic acid labeling, fluorescence dyes or labels, intermediate binding partners such as biotin, etc.) that facilitates their specific detection after being deposited onto a planar surface. The use of multiple types of tracer cells, distinguished from each other by the labeling method (e.g. different stain colors or fluorescent moieties) or some physical characteristic (e.g. size, shape) is also disclosed. Depending on the application, the tracer cells can be used by themselves or mixed with other non-labeled cells, cultured cells, or clinical specimens. The use of tracer cells as a cell-based internal control is also envisioned as one embodiment of this invention. The detection method and quantitative assessment of cell deposition and distribution depends on the method used to label the tracer cells and can range from manual visualization of the cells under a microscope to image acquisition instruments with associated software algorithms.

Although a variety of methods are available to label tracer cells in order to detect them and distinguish them from other cells, one embodiment of the present invention is to simply label them with a nuclear stain, such as hematoxylin. For example, CaSki tracer cells may be easily labeled in solution with hematoxylin by suspending the cells in a phosphate-buffered saline (PBS) solution, incubating the cells with a hematoxylin stain at room temperature for 15 to 30 minutes with gentle agitation, washing the cells in PBS to remove excess stain, and storing the cells in either PBS or PreservCyt® Solution (Cytyc Corporation, Boxborough, Mass.).

To monitor the effectiveness of the ThinPrep® system, tracer cells can be used alone, in conjunction with unstained cultured cells (CaSki or another cell line(s)) or added to clinical specimens. In one embodiment of the invention, only the tracer cells are labeled and thus are the only cells easily visualized. For example, if hematoxylin-stained CaSki tracer cells are added to a clinical specimen, the subsequently produced ThinPrep® slide would simply be fixed and coverslipped prior to evaluation. These slides would not be stained according to the normal procedure because that protocol also includes hematoxylin which would obscure the tracer and clinical cells. However, the non-stained cells from the clinical specimen could be viewed by phase-contrast microscopy. It is anticipated that tracer cells can be labeled by other stains or methods that would allow them to be distinguished from a background of clinical cells stained conventionally with a Pap (hematoxylin) stain.

One advantage of this embodiment is that hematoxylin-stained tracer cells on glass slides can be easily visualized macroscopically by eye (if the density is great enough), manually using a microscope or automatically using an image acquisition system and associated software such as the ThinPrep® Imaging System (Cytyc Corporation, Boxborough, Mass.), CountCell software program and Kaleido-Spot software program which provides precise quantitative and spatial information.

The ThinPrep® Imaging System is an example of imaging device that has been designed to detect Pap-stained cervical cells on glass microscope slides. Therefore, the ThinPrep® Imaging System could be used to detect hematoxylin-stained CaSki tracer cells. Multiple, overlapping images are acquired in order to image the entire cell spot (cells applied to the microscope slide using the ThinPrep® 2000 or ThinPrep® 3000 processor).

The x-y coordinates of every object (primarily individual tracer cells but also small cell clusters) are recorded to an output file along with the area of the object. The CountCell program (written in C++) is then used to: a) eliminate redundant objects with common x-y coordinates; b) calculate the number of tracer cells per object (divide area of object by average area of a CaSki cell); c) map objects to a 56×50 array (each grid in the array measuring 0.5 mm×0.5mm); d) sum the number of tracer cells per grid in the array; e) establish the cell spot border; and f) create a tab-delimited output file of the cell counts in the 56×50 array. The Kaleido-Spot program (written in Excel Visual Basic) then uses the CountCell output to calculate the total number of cells in the cell spot, mean cell count per grid, correlation of variance across the cell spot as a measure of variability, number of cells on the edge of the cell spot and the percentage of cells on the edge of the cell spot. In addition to these calculations, a number of tools are provided within Kaleido-Spot to examine the distribution of cells, visualize the cellular uniformity, and locate holes or cell clusters according to size and cell density.

In another embodiment of the present invention, labeled tracer cell(s) may be added to sample cells so that the behavior of the sample cells in the presence or absence of different variables may be indirectly monitored without influencing the behavior of the samples cells. For example, labeled tracer cell(s) may be added to sample cells and used to monitor the efficiency of cell transfer to a microscope slide in the presence or absence of blood, mucus or other biological components of the collected cell specimen. Similarly, the effect of non-biological materials that may potentially contaminate and interfere with the collection, transfer and visualization of cells may be studied. For example, the effect of lubricants, vaginal creams, douches, or spermacides on the preparation of cytology slides from cervical specimens may be systematically evaluated. In addition, improvements to the process of depositing cells on surfaces may be systematically explored. For example, within the ThinPrep system, the effect on cell transfer and distribution may be studied with respect to mechanical or software changes to the processor or modifications to system components including alternative formulations of preservative solution, different filter membrane materials, treatments or properties and different glass slide surface properties. Experiments demonstrating the effects of different variables on cell samples are shown below in the Examples section.

In still yet another embodiment, a method for determining the cell retention properties of a planar surface comprising the steps of labeling a plurality of tracer cells such that each plurality of tracer cells may be distinguished from one another, adding the labeled tracer cells to specimen cells in liquid suspension to form a mixed cell sample, transferring the mixed cell sample onto a planar surface, washing the planar surface, and determining the number and/or type of tracer cells retained on the planar surface wherein the number and/or type of tracer cells retained on the planar surface is indicative of the cell retention properties of the planar surface, is presented. Examples of a planar surface may be a microscope slide, microtiter dish, or a filter membrane. The mixed cell sample contains labeled or stained tracers cells or populations of tracer cells as well as containing labeled or unlabeled sample cells. The sample cells may be cervical cells. The tracer cells and/or sample cells may be labeled with a variety of stains or other detection moieties such as cytological stains, a radioactive moiety, chromophores, fluorescent moieties, nucleic acid stains, immunological reagents, or intermediate binding partner and/or immunohistological stains.

In yet another embodiment of the present invention, the use of labeled tracers cells may be used as an indirect measurement of cellularity (i.e., concentration of cells in a sample), spatial orientation of cells on a planar surface (e.g., microscope slide), cell size or shape (for filtration purposes), or the biochemical composition of a cell (membrane constituency). For example, by differentially labeling tracer cells that differ in size or surface characteristics (e.g. label large tracer cells with hematoxylin and small tracer cells with Brazilian red) the functional performance of filters that differ in material, surface charge, pore size, pore density or other variables can be assessed by detecting which tracer cells are effectively retained on the filter after filtration.

Tracer cells having the identical mode of staining or labeling as the sample cells may be placed on a microscope slide to assist in determining the spatial orientation of the labeled sample cells. For example, once sample cells have been prepared and stained, the cells may be manually visually inspected by a cytotechnologists, typically under magnification, and with or without various sources of illumination. An automated processor, such as the ThinPrep® 2000 Processor (Cytyc Corporation, Boxborough, Mass.) may be used to collect cells from a cell collection container and deposit them in a thin layer on a glass slide for analysis.

Additionally, automated machine vision systems have been adapted to aid cytological inspection. The cells may also be selectively stained with an antibody specific to a protein of interest. The antibody has also been conjugated with a fluorescent moiety so that the fluorescent label emits a light in the visual range when excited by light of a specific wavelength. Tracers cells having been labeled with the same fluorescent moiety as the sample cells can be placed onto a microscope slide at a predetermined location either before or during the processing of the sample cells. By placing the labeled tracer cells onto the microscope side at a known location, an automated machine vision system may locate the spatial orientation of the tracer cells and hence the sample cells by detecting the light emitted by the fluorescent label. Thus, the labeled tracer cells act as a fiducial mark for cells containing selective staining or labeling of a macromolecular species.

Finally, it is anticipated that tracer cells can be used in many of the applications described above without having been pre-labeled by any method. In one embodiment, tracer cells with a distinct characteristic—for example a detectable cell surface protein unique to these tracer cells—can be added to non-labeled cells or sample cells and deposited onto a surface. The tracer cells can then be detected by standard immunocytochemistry methods using an antibody specific to the tracer cell membrane protein.

### EXAMPLES

### Example 1

### Interference by Lubricants

Objective:

The objective was to determine the effect of various lubricant formulations in the ThinPrep® 2000 System.

Design:

Samples were prepared in 20 ml (final volume) of PreservCyt® Solution that contained 360,000 CaSki cells and 40,000 pre-stained CaSki tracer cells. The “test” samples also contained 100 mg of lubricant. Three different lubricant formulations, designated “A”, “B”, and “C”, from the same manufacturer were examined. Test samples were prepared by adding 1 ml of 10% lubricant stock solutions (prepared in PreservCyt® Solution) to 19 ml of the cell mixture. Control samples, containing no lubricant, were prepared by adding 1 ml of PreservCyt® Solution to 19 ml of the cell mixture.

Slides were prepared from each sample using the ThinPrep® 2000 processor. After preparation, slides were exposed to 95% alcohol and xylene and cover-slipped without any further cell staining. Slides were then imaged on the ThinPrep® Imaging System and tracer cell counts obtained from the data using the CellCount and Kaleido-Spot software programs.

Results:

TABLE 1

| | Tracer | | |
|---|---|---|---|
| Lubricant | Cells per | | Uniformity* |
| (100 mg) | Slide | % Control | (% CV) |
| No Lubricant | 30,006 | 100 | 69 |
| (Control) | | | |
| Lubricant “A” | 10,907 | 36 | 69 |
| Lubricant “B” | 7,798 | 26 | 78 |
| Lubricant “C” | 5,497 | 18 | 99 |

*Measure of the distribution of cells on the slide; percent coefficient of variation of cell numbers per 0.5 mm × 0.5 mm grid.

About 30,000 tracer cells were present on the control slide. Sub-dividing the cell spot area of the slide into about 1500 grids, each 0.5 mm×0.5 mm square, showed that the cells were distributed among all grids with a 69% CV.

As can be seen in the Table 1, each of the three lubricants interfered with the preparation of slides. For example, a sample containing 100 mg of lubricant “A” produced a slide with 10,907 cells, or 36% of the cell number as on the control slide without lubricant. However, the distribution of these cells was similar to that of the control. Slides prepared from samples containing Lubricants “B” and “C” had both fewer cells and less even distribution of those cells than the control.

Conclusion:

The data permitted a quantitative comparison between the various lubricant formulations.

For example, lubricant “A” resulted in twice the number of cells than lubricant “C”.

Although each of these lubricants perturbed the preparation of slides in the ThinPrep system, lubricant “A” had the least deleterious effects.

### Example 2

### Interaction of Preservative Formulation and Blood

Objective:

The objective was to determine the performance of the ThinPrep System for preparing slides from samples prepared in two different formulations of cell preservative and in the absence or presence of blood.

Experimental Design:

Samples were prepared in 20 ml (final volume) of either cell preservative “A” or “B” that contained 300,000 CaSki cells and 100,000 pre-stained CaSki tracer cells. 100 ul of human blood (citrate treated to prevent coagulation) was added to half of the samples. Slides were prepared from each sample using the ThinPrep® 2000 processor. After preparation, slides were exposed to 95% alcohol and xylene and cover-slipped without any further cell staining. Slides were then imaged on the ThinPrep® Imaging System and tracer cell counts obtained from the imaged data using the CellCount and Kaleido-Spot software programs.

Results:

TABLE 2

Blood

| | | No | Yes |
|---|---|---|---|
| Cell | “A” | 20,880 | 639 |
| Preservative | “B” | 35,619 | 4,851 |
| Formula | | | |

In the absence of blood, cell preservative “B” produced slides containing an average of 35,619 cells, or roughly 1.7 times more cells than preservative “A” (20,880 cells) (see Table 2). The addition of blood had a pronounced affect on both cell preservative formulations. In the presence of blood, slides contained only 3% or 14% the number of cells as in the absence of blood, for cell preservatives “A” and “B”, respectively. However, even in the presence of blood, cell preservative “B” performed better than preservative “A” as it generated slides with an average of 4,851 cells, or 7.6 times the amount of cells as preservative “A”.

Conclusion:

These data clearly showed the advantages of the cell preservative “B” formulation for the optimization of slide preparation, particularly in cases where blood is present.

### Example 3

### Reproducibility of Preparing Multiple Successive Slides

Objective:

The objective was to determine the consistency of slides prepared sequentially from the same specimen.

Experimental Design:

A 20 ml sample was prepared in PreservCyt® Solution. This sample was comprised of a pool of residual clinical specimens containing about 1,600,000 epithelial cells and 600,000 pre-stained CaSki tracer cells.

This 20 ml cell mixture was dispensed into a specimen vial and a slide prepared on the ThinPrep® 2000 processor. The volume remaining in the vial was measured and recorded. The volume necessary to produce the slide was calculated (20 ml minus the remaining volume) and this volume of PreservCyt® Solution was added to the vial to restore the sample volume to 20 ml.

A second slide was then produced on the ThinPrep® 2000 processor and the remaining volume measured and recorded. As before, the volume of sample removed by the processor was replaced with fresh PreservCyt® Solution. This process continued until the ThinPrep® processor generated a “Sample is Dilute” error message, meaning a slide could not be prepared with at least 25% of the cell density as a normal slide.

During this process, the cell concentrations became increasingly dilute as a consequence of cell removal and replacement by PreservCyt® Solution. In turn, the volume required to make each sequential slide increased.

Each slide was exposed to 95% alcohol and xylene and cover-slipped without any further cell staining. The number of tracer cells on each slide was determined by imaging the slides on the ThinPrep® Imaging System and processing the data using the CountCell and Kaleido-Spot software programs.

Results:

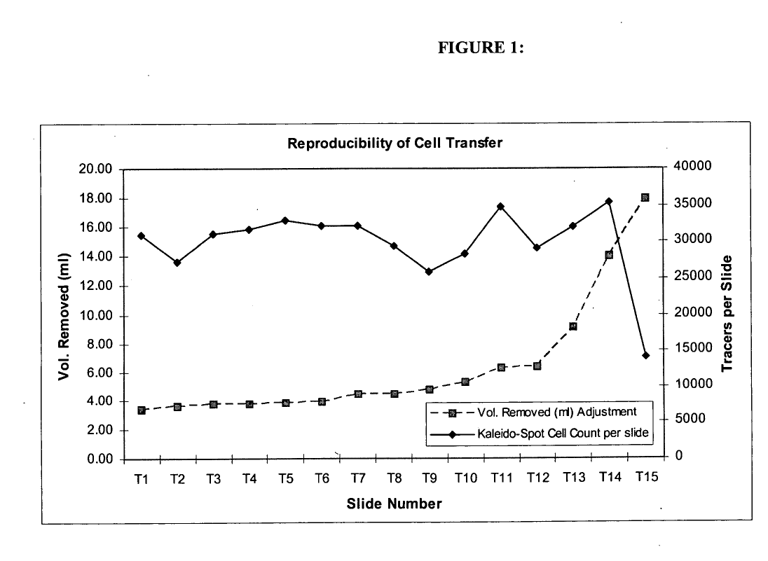

A total of 15 slides were produced in succession from the original epithelial/tracer cell sample. The ¹⁵th slide produced the “Sample is Dilute” error message on the ThinPrep® 2000 processor. FIG. 1 shows a plot of the volume required to make each slide and the tracer cell count on each slide for the 15 consecutive slides.

The FIG. 1 clearly shows that the number of tracer cells, and by inference the number of epithelial cells, was relatively constant for the first 14 successive slides. The mean tracer cell count was 31,000 cells per slide with only an 8% CV across the series. As expected from the instrument error code, the last slide had far fewer cells than the rest, with only about 14,000 tracer cells.

These data clearly indicate that the ThinPrep® processor produced slides with a consistent number of cells on each slide. Each slide contained about 31,000 tracer cells and, by calculation, 83,000 epithelial cells (since each slide contained 31,000 tracer cells, or 5.17% of the input, then they should also contain 5.17% of the epithelial cell input; 0.0517×1,600,000=82,667).

As expected, the volume required to produce each successive slide increased. However, the increase in sample volume for each slide was modest through the first 12 slides, but then increased rapidly. For example, 3.4 ml were removed for the first slide and 5.3 ml for the 10^{th} slide. By the 14^{th} slide, about 14 ml of sample were required to make a slide, although the cell counts were similar to all the previous 13 slides. Although the cellular concentration of the sample for the 14ₜₕ slide was only about 8% that of the original sample, both the 1^{st} and 14^{th} slides contained similar amounts of cells.

The processor removed as much volume as possible to produce the last slide (greater than 18 ml; there is always residual material left over due to the “dead volume” at the very bottom of the vial which is inaccessible to collection because of physical, instrument design constraints), but by this point there were not enough cells to create a slide with “normal” cell density.

Conclusion:

In summary, 14 slides with similar amounts of cells (only 8% CV) were consecutively produced on the ThinPrep® 2000 instrument from the same sample. These data showed that the ThinPrep® 2000 processor reproducibly produced similar slides. Furthermore, since the cell concentration in the sample progressively became more dilute, these data showed that the ThinPrep processor was able to effectively monitor, collect and transfer similar numbers of cells to slides regardless of cell concentration in the sample.

### Example 4

### Wash Treatment of Specimens

Objective:

The objective was to determine if the process of washing clinical specimens with a mixture of cell preservative and glacial acetic acid could mitigate the interference of lubricants in the preparation of specimen slides.

Experimental Design:

Sixteen identical samples were prepared; each contained about 1,000,000 epithelial cells and 100,000 pre-stained CaSki tracer cells in 20 ml (final volume) of PreservCyt® Solution. In addition, 4 samples contained 50 mg of a lubricant, 4 samples contained 105 mg of a lubricant and 4 samples contained 200 mg of a lubricant. The final 4 samples were controls and did not contain any lubricant.

Two samples from each lubricant group were washed with CytoLyt™ (Cytyc Corporation, Boxborough, Mass.) plus glacial acetic acid process. Briefly, the cells were pelleted by centrifugation at 1200×g for 5 minutes, the PreservCyt® Solution was removed and 30 ml of CytoLyt™ Solution plus glacial acetic acid was added. After mixing briefly, the cells were centrifuged as before, the wash solution removed, and the cells re-suspended in 20 ml of fresh PreservCyt Solution. These were the test samples.

The other two samples from each lubricant group did not undergo the wash process.

These were the control samples.

Slides were subsequently prepared from each sample using the ThinPrep® 2000 processor. After preparation, slides were exposed to 95% alcohol and xylene and cover-slipped without any further cell staining. Slides were then imaged on the ThinPrep® Imaging System and tracer cell counts obtained from the data using the CellCount and Kaleido-Spot software programs.

Results:

TABLE 3
Effect of Wash Treatment on Specimens Containing Lubricant

| Lubricant | Control (No Wash) | Test (Wash Treatment) |
|---|---|---|
| Amount | Average Tracer Cells | Average Tracer Cells |
| (mg) | per Slide | Per Slide |
| 0 | 10,271 | 16,380 |
| 50 | 4,837 | 15,697 |
| 105 | 3,118 | 19,190 |
| 200 | 1,937 | 17,178 |

Increasing amounts of lubricant in the control samples—those that were not treated with the wash process—resulted in decreasing amounts of cells on the slides (see Table 3). In the absence of lubricant, there was an average of about 10,300 cells per slide. However, in the presence of 50 mg, 105 mg and 200 mg of lubricant, the slides contained only about 4800, 3100 and 1900 cells, respectively. At the highest level of lubricant tested, the resulting slides had only about 19% of the cell numbers as the no-lubricant control.

The results of the samples that underwent the wash process prior to slide processing in the ThinPrep® 2000 instrument were very different. After treatment of samples containing from 0-200 mg of lubricant with the wash process, all slides contained similar numbers of cells, ranging from about 15,700 to 19,200. These data clearly indicate that the wash process is effective for removing the interference of up to 200 mg of lubricant.

Greater numbers of cells were found to be on the post-wash no-lubricant slides than on the no-lubricant control slides. Using the tracer cell tool in this experiment, this phenomenon could be quantified. The wash process yielded an approximately 60% increase in cell numbers, from about 10,300 cells per slide without washing the cells to about 16,400 cells per slide following the pre-processing wash procedure.

Conclusion:

The process of washing specimens with glacial acetic acid in CytoLyt was effective at removing the interference of lubricant in amounts up to 200 mg per specimen.

Additionally, even in the absence of lubricant, greater numbers of cells were present on slides following the washing process.

Equivalents

Those skilled in the art will recognize, or be able to ascertain using no more than routine experimentation, many equivalents to the specific embodiments of the invention described herein. The invention can be embodied in other specific forms without departing from the spirit or essential characteristics thereof. The present embodiments are therefore to be considered in all respects as illustrative and not restrictive. The scope of the invention is indicated by the appended claims, rather than by the foregoing description, and all changes which come within the meaning and range of equivalency of the claims are therefore intended to be embraced therein.

1. A method for determining the deposition of cells onto a planar surface, the method comprising the steps of:
a. labeling tracer cells;
b. adding said labeled cells to a collection of sample cells that are in liquid suspension;
c. dispersing said sample cells onto to a planar surface;
d. determining the effectiveness of transfer of the sample cells by determining the number of labeled cells transferred to the planar surface.

2. The method of claim 1 wherein the planar surface is a microscope slide.

3. The method of claim 1 wherein the liquid suspension contains unlabeled cells.

4. The method of claim 1 wherein the sample cells are cervical cells.

5. The method of claim 1 wherein the liquid suspension contains a cell preservative.

6. The method of claim 1 wherein the tracer cells are labeled with a cytological stain, a radioactive moiety, a chromophore, a fluorescent moiety, a nucleic acid stain, an immunological reagent, an intermediate binding partner and/or immunohistological stains.

7. A method for determining the effectiveness and uniformity of cells deposited onto a planar surface, the method comprising the steps of:
a. labeling tracer cells;
b. adding said labeled cells to a collection of sample cells that are in liquid suspension;
c. transferring the sample cells onto a microscope slide;
d. determining the effectiveness and uniformity of transfer of the sample cells by determining the number and distribution of labeled cells transferred to the microscope slide.

8. The method of claim 6 wherein the planar surface is a microscope slide.

9. The method of claim 6 wherein the liquid suspension contains unlabeled cells.

10. The method of claim 6 wherein the sample cells are cervical cells.

11. The method of claim 6 wherein the liquid suspension contains a cell preservative.

12. The method of claim 6 wherein the tracer cells are labeled with a cytological stain, a radioactive moiety, a chromophore, a fluorescent moiety, a nucleic acid stain, an immunological reagent, an intermediate binding partner and/or immunohistological stains.

13. The method of claim 6 wherein determining the effectiveness and uniformity of transfer of the unlabeled cells to the microscope slide is calculated by an algorithm based on the labeled cells.

14. A method of determining the focal plane of a planar surface, the method comprising the steps of:
a. labeling tracer cells;
b. adding said labeled cells to a collection of sample cells that are in liquid suspension;
c. transferring the sample cells onto a planar surface;
d. determining the focal plane of the planar surface by determining the location of the labeled cells on the planar surface.

15. The method of claim 11 wherein the tracer cells are labeled with a cytological stain, a radioactive moiety, a chromophore, a fluorescent moiety, a nucleic acid stain, an immunological reagent, an intermediate binding partner and/or and immunohistological stains.

16. The method of claim 11 wherein the planar surface is a microscope slide.

17. The method of claim 11 wherein the liquid suspension contains unlabeled cells.

18. The method of claim 11 wherein the sample cells are cervical cells.

19. The method of claim 11 wherein the liquid suspension contains a cell preservative.

20. A method for determining the cell retention properties of a planar surface, the method comprising the steps of:
a. labeling a plurality of tracer cells such that each plurality of tracer cells may be distinguished from one another,
b. adding said labeled tracer cells to sample cells in liquid suspension to form a mixed cell sample,
c. transferring said mixed cell sample onto a planar surface,
d. washing said planar surface, and
e. determining the number and/or type of tracer cells retained on said planar surface;
wherein the number and/or type of tracer cells retained on said planar surface is indicative of the cell retention properties of said planar surface.

21. The method of claim 20 wherein said planar surface is a microscope slide.

22. The method of claim 20 wherein said planar surface is a filter membrane.

23. The method of claim 20 wherein said mixed cell sample contains unlabeled cells.

24. The method of claim 20 wherein said sample cells are cervical cells.

25. The method of claim 20 wherein said liquid suspension contains a cell preservative.

26. The method of claim 20 wherein said tracer cells are labeled with a cytological stain, a radioactive moiety, a chromophore, a fluorescent moiety, a nucleic acid stain, an immunological reagent, an intermediate binding partner and/or immunohistological stains.
